(19) United States
(12) Patent Application Publication
(10) Pub. No.: US 2019/0112599 A1
(43) Pub. Date: Apr. 18, 2019

(54) Methods and Compositions for the Single Tube Preparation of Sequencing Libraries Using Cas9

(71) Applicant: President and Fellows of Harvard College, Cambridge, MA (US)

(72) Inventors: George M. Church, Brookline, MA (US); Benjamin W. Pruitt, Cambridge, MA (US); Richard C. Terry, Carlisle, MA (US)

(21) Appl. No.: 16/088,867

(22) PCT Filed: Mar. 29, 2017
(86) PCT No.: PCT/US17/24662

§ 371 Date: Sep. 27, 2018

Related U.S. Application Data
(60) Provisional application No. 62/315,751, filed on Mar. 31, 2016.
(60) Provisional application No. 62/321,890, filed on Apr. 13, 2016.

(51) Int. Cl. C12N 15/10; C12N 15/113; C40B 50/06; C12Q 1/686; C12Q 1/6806; C12Q 1/6869; C12N 9/00

(52) U.S. Cl. CPC C12N 15/1093; C12N 15/113; C40B 50/06; C12Q 1/686; C12Y 600/00; C12Q 1/6869; C12N 9/93; C12N 2330/31; C12N 2310/20; C12Q 1/6806

(57) ABSTRACT

Methods and compositions of single tube preparation of sequencing libraries from a target DNA are provided. The methods include contacting the DNA with a composition comprising Cas9 endonuclease, a first and a second guide RNAs, a ligase, and sequencing adapters, subjecting the composition to thermal cycling to cleave the DNA at the sites flanking the regions of interest by the RNA guided endonuclease, and subjecting the composition to a temperature to allow ligation of the cleaved DNA fragments including the regions of interest with the sequencing adapters to generate the sequencing libraries.

### RELATED APPLICATION DATA

This application claims priority to U.S. Provisional Application No. 62/315,751 filed on Mar. 31, 2016 and to U.S. Provisional Application No. 62/321,890 filed on Apr. 13, 2016 which are hereby incorporated herein by reference in their entirety for all purposes.

### FIELD

The present invention relates in general to methods and compositions for the single tube preparation of sequencing libraries using Cas9.

### BACKGROUND

The CRISPR type II system is a recent development that has been efficiently utilized in a broad spectrum of species. See Friedland, A. E., et al., Heritable genome editing in C. elegans via a CRISPR-Cas9 system. Nat Methods, 2013. 10(8): p. 741-3, Mali, P., et al., RNA-guided human genome engineering via Cas9. Science, 2013. 339(6121): p. 823-6, Hwang, W. Y., et al., Efficient genome editing in zebrafish using a CRISPR-Cas system. Nat Biotechnol, 2013, Jiang, W., et al., RNA-guided editing of bacterial genomes using CRISPR-Cas systems. Nat Biotechnol, 2013, Jinek, M., et al., RNA-programmed genome editing in human cells. eLife, 2013. 2: p. e00471, Cong, L., et al., Multiplex genome engineering using CRISPR/Cas systems. Science, 2013. 339(6121): p. 819-23, Yin, H., et al., Genome editing with Cas9 in adult mice corrects a disease mutation and phenotype. Nat Biotechnol, 2014. 32(6): p. 551-3. CRISPR is particularly customizable because the active form consists of an invariant Cas9 protein and an easily programmable guide RNA (gRNA). See Jinek, M., et al., A programmable dual-RNA-guided DNA endonuclease in adaptive bacterial immunity. Science, 2012. 337(6096): p. 816-21. Of the various CRISPR orthologs, the Streptococcus pyogenes (Sp) CRISPR is the most well-characterized and widely used. The Cas9-gRNA complex first probes DNA for the protospacer-adjacent motif (PAM) sequence (—NGG for Sp Cas9), after which Watson-Crick base-pairing between the gRNA and target DNA proceeds in a ratchet mechanism to form an R-loop. Following formation of a ternary complex of Cas9, gRNA, and target DNA, the Cas9 protein generates two nicks in the target DNA, creating a blunt double-strand break (DSB) that is predominantly repaired by the non-homologous end joining (NHEJ) pathway or, to a lesser extent, template-directed homologous recombination (HR). CRISPR methods are disclosed in U.S. Pat. Nos. 9,023,649 and 8,697,359. The RNA-guided endonuclease CRISPR/Cas9 system has been established with proven usefulness in a wide variety of in vivo applications, from mammalian genome editing to artificially-skewed allelic selection. As next-generation sequencing is increasingly used as a clinical diagnostic tool, there remains a need for the development of simple, low-cost targeted library preparation pipelines.

### SUMMARY

The present disclosure provides for a novel in vitro technique that harnesses the highly configurable nature of Cas9-mediated DNA cutting to enable rapid, single-tube next-generation sequencing library preparation. Unlike existing targeted library preparation techniques, the presently disclosed Cas9-mediated pipeline requires no initial PCR and can take place in a single tube. Briefly, DNA isolate is added to a solution containing Cas9, guide RNAs designed to flank regions of interest (e.g., common oncogenes), thermophilic DNA ligase, and sequencing adapters. Subsequent thermal cycling catalyzes initial cutting of the targeted regions of interest followed by temperature-dependent ligation of the relevant sequencing adapters (e.g., Illumina sequencing adapters). The result is an adapter-ligated sequencing library comprised of the targeted regions of interest, requiring no additional size selection or, in many cases, error-prone amplification. Not only does this technique combine the costly and time consuming selection, enrichment, and library preparation steps into a single reaction, but it also allows for a fully PCR-free sequencing pipeline, which is highly desirable in the context of single nucleotide polymorphism (SNP)-detection and other error-sensitive clinical applications.

The present disclosure provides a method of preparing a sequencing library from a target DNA comprising the steps of contacting the DNA with a composition comprising an endonuclease, a first guide RNA, a second guide RNA, a ligase, and sequencing adapters, wherein the first and second RNAs guide the endonuclease to specific sites flanking regions of interest in the DNA, subjecting the DNA and the composition to thermal cycling to allow cleavage of the DNA at the sites flanking the regions of interest by the endonuclease, and subjecting the DNA and the composition to a temperature to allow ligation of the cleaved DNA fragments including the regions of interest with the sequencing adapters to generate a sequencing library.

The present disclosure further provides a method of determining a sequence of interest in a target DNA comprising the steps of contacting the DNA with a composition comprising an endonuclease, a first guide RNA, a second guide RNA, a ligase, and sequencing adapters, wherein the first and second RNAs guide the endonuclease to sites flanking the sequence of interest in the DNA, subjecting the DNA and the composition to thermal cycling to allow cleavage of the DNA at sites flanking the sequence of interest by the endonuclease, subjecting the DNA and the composition to a temperature to allow ligation of the cleaved DNA fragment including the sequence of interest with the sequencing adapters to generate a ligation product, and sequencing the ligation product to determine the sequence of interest.

The present disclosure provides a composition for preparing a sequencing library from a target DNA comprising a first enzyme comprising an endonuclease, a first nucleotide sequence comprising a first guide RNA, a second nucleotide sequence comprising a second guide RNA, a second enzyme comprising a ligase, and a buffer comprising a solution in which both the endonuclease and ligase are active. The composition according to the disclosure further comprises a third nucleotide sequence (or pair of sequences) comprising a first sequencing adapter and a fourth nucleotide sequence (or pair of sequences) comprising a second sequencing adapter,

The present disclosure further provides a kit for preparing a sequencing library from a target DNA comprising the composition of the disclosure, and a reagent for reconstitution and/or dilution.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIGS. 1A-C depict a process overview. FIG. 1A shows that the minimum reaction is constituted by: double stranded target DNA (genomic/plasmid/synthetic), Cas9 pre-complexed with one or more pairs of fragmentation gRNAs, a thermophilic DNA ligase, and application-specific adapter oligonucleotides. All components are present for all reaction steps (diagram B simplified for clarity). FIG. 1B shows that the process involves four sequential steps, delineated by temperature. FIG. 1C shows that at 37° C., the pre-complexed Cas9-gRNA holoenzymes catalyze the selective fragmentation of the target DNA. Denaturation at 95° C. removes Cas9 from the fragmented DNA and subsequent cooling allows for the nucleic acids to properly anneal. Continuation of the reaction at 45° C. allows the thermophilic ligase to catalyze the ligation of adapter oligonucleotides onto the DNA fragments.

FIG. 2 shows that single tube Cas9 library preparation provides SNP detection comparable to direct PCR-based library preparation. E. coli MG1655 genomic DNA extracted from a population of cells resistant to the antibiotic rifampicin was subjected to both a traditional targeted PCR-based library preparation pipeline and a single tube Cas9-based library preparation pipeline. There are well-characterized mutations in the rpoB gene that confer resistance to rifampicin, and next-generation sequencing is a common means of determining the identity and frequency of these mutations at a population level. (n=5 independent technical replicates, error bars are S.E.M.)

### DETAILED DESCRIPTION

Embodiments of the present disclosure are directed to methods and compositions of single tube preparation of sequencing libraries using Cas9. Cas9 is an RNA-guided endonuclease that can be used in vitro to cleave DNA molecules. Prior publications/inventions describe multiple ways in which Cas9 may be used to fragment or otherwise excise target DNA prior to use in downstream assays. The present disclosure provides a single tube/single reaction method for the preparation of next generation sequencing libraries. In short, a mixture of Cas9 (pre-complexed with gRNAs), a thermophilic DNA ligase (e.g., 9oN), and adapter oligonucleotides are mixed with target DNA (e.g., human genomic DNA). Targeted Cas9 cleavage proceeds at 37° C., producing short fragments with ends amenable to ligation. Following cleavage, heat denaturation at 95° C. removes Cas9 from the fragment ends. Cooling to 45° C. allows for renaturation of the target DNA followed by ligation of adapter oligos. The resulting mixture is then suitable for direct use in indexing PCR reactions, or, following purification, direct use on sequencing instruments.

The disclosure further provides kits derived from this concept that can be distributed as single solution mixtures that can be used for in vitro library preparations (i.e., requiring only the direct addition of human genomic DNA to the kit solution) or for in situ library preparations (i.e., in which the reagent(s) of the kit may be applied directly to fixed cells or tissue samples). In the case of in situ library preparations, the resulting adapter ligated DNA can be amplified by an in situ PCR method such as polony PCR (within an acrylamide gel), in which case the original spatial location of the target genomic DNA may be preserved. Relative to other, similar library preparation workflows, the presently disclosed method requires no intermediate steps or liquid handling beyond the initial addition of genomic DNA. With the latest advances in patterned flowcell technologies (that allow for the direct loading of sequencing libraries at any concentration), libraries prepared using this method can potentially be directly loaded onto a sequencing device. The disclosure provides kits containing gRNAs targeting a panel or pathway of genes (e.g., breast cancer oncogenes), which can dramatically reduce the costs and time associated with clinical sample handling.

The disclosure provides this general approach which works with any nucleic-acid guided or programmable endonuclease that can be heat inactivated at 98° C. This includes but is not limited to: Cas9 orthologs (e.g., NM-Cas9, ST1-Cas9), engineered Cas9 variants (e.g., eCas9, Cas9-HF1), and other cas family RNA-guided endonucleases (e.g., Cpf1). Cas9 variants and orthologs provide means of addressing a larger target site space. Various Cas9 orthologs and variants are known in the art as described in Esvelt K M et al., “Orthogonal Cas9 proteins for RNA-guided gene regulation and editing”, Nature Methods, 2013, Vol. 10, pages 1116-1121; Mali P. et al., “RNA-guided human genome engineering via Cas9”, Science, 2013, Vol. 339(6121):823-6, Epub 2013 Jan. 3; Zetsche et al., “Cpf1 Is a Single RNA-Guided Endonuclease of a Class 2 CRISPR-Cas System”, Cell, 2015, Vol. 163, Issue 3, p′759-771, Mali P. et al., “Cas9 as a versatile tool for engineering biology”, Nature Methods, 2013, Vol. 10, pages 957-963, the contents of which are incorporated herein in their entireties.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The present disclosure provides a method of preparing a sequencing library from a target DNA comprising the steps of contacting the DNA with a composition comprising an endonuclease, a first guide RNA, a second guide RNA, a ligase, and sequencing adapters, wherein the first and second RNAs guide the endonuclease to specific sites flanking regions of interest in the DNA, subjecting the DNA and the composition to thermal cycling to allow cleavage of the DNA at the sites flanking the regions of interest by the endonuclease, and subjecting the DNA and the composition to a temperature to allow ligation of the cleaved DNA fragments including the regions of interest with the sequencing adapters to generate a sequencing library.

Embodiments of the disclosure provide “adapter sequences”, “adapter oligos” or “adapters” which are generally oligonucleotides of at least 5, 10, or 15 bases and preferably no more than 50 or 60 bases in length; however, they may be even longer, up to 100 or 200 bases. Adapter sequences/oligos may be synthesized using any methods known to those of skill in the art. For the purposes of this invention they may, as options, comprise primer binding sites, recognition sites for endonucleases, common sequences and promoters. The adapter may be entirely or substantially double stranded or entirely single stranded. A double stranded adapter may comprise two oligonucleotides that are at least partially complementary. The adapter may be phosphorylated or unphosphorylated on one or both strands.

Adapters as contemplated by the disclosure may also incorporate modified nucleotides that modify the properties of the adapter sequence/oligo. For example, phosphorothioate groups may be incorporated in one of the adapter strands. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified backbone of an S-Oligo is resistant to the action of most exonucleases and endonucleases. Phosphorothioates may be incorporated between all residues of an adapter strand, or at specified locations within a sequence. A useful option is to sulfurize only the last few residues at each end of the oligo. This results in an oligo that is resistant to exonucleases, but has a natural DNA center.

In one embodiment, the target DNA is mammalian genomic DNA. In another embodiment, the target DNA is human genomic DNA. In one embodiment, the target DNA is bacterial genomic DNA. In another embodiment, the target DNA is synthetic DNA. In one embodiment, the synthetic DNA is in the form of transfected or integrated library.

In one embodiment, the first and second guide RNAs are complementary to sequences flanking the regions of interest in the DNA. In one embodiment, the endonuclease comprises Cas9, Cas9 orthologs or engineered Cas9 variants. In another embodiment, the Cas9 orthologs comprise NM-/ST1-Cas9 and Cpf1. In yet another embodiment, the engineered Cas9 variants comprise eCas9 and Cas9-HF1.

In one embodiment, the sequencing adapters are added to 5′ and 3′ ends of the cleaved DNA fragments by ligation. In one embodiment, the ligase is a thermophilic DNA ligase. In one embodiment, a plurality of sequencing libraries are prepared from a plurality of target DNAs. In one embodiment, the steps are performed directly in a cell culture or tissue sample and the resulting sequencing libraries are amplified by in situ PCR. In another embodiment, the cell and tissue samples are fixed.

The present disclosure further provides a method of determining a sequence of interest in a target DNA comprising the steps of contacting the DNA with a composition comprising an endonuclease, a first guide RNA, a second guide RNA, a ligase, and sequencing adapters, wherein the first and second RNAs guide the endonuclease to sites flanking the sequence of interest in the DNA, subjecting the DNA and the composition to thermal cycling to allow cleavage of the DNA at sites flanking the sequence of interest by the endonuclease, subjecting the DNA and the composition to a temperature to allow ligation of the cleaved DNA fragment including the sequence of interest with the sequencing adapters to generate a ligation product, and sequencing the ligation product to determine the sequence of interest.

Embodiments of the disclosure provide methods of ligation. Methods of ligation will be known to those of skill in the art and are described, for example in Sambrook et at. (2001) and the New England BioLabs catalog both of which are incorporated herein by reference for all purposes. Methods of ligation contemplated by the disclosure can be based on using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid accepter through the formation of a 3′→5′ phosphodiester bond, substrates include single-stranded RNA and DNA as well as dinucleoside pyrophosphates; or any other methods described in the art. Fragmented DNA may be treated with one or more enzymes, for example, an endonuclease, prior to ligation of adapters to one or both ends to facilitate ligation by generating ends that are compatible with ligation. In an exemplary embodiment, a thermophilic DNA ligase is used. The thermophilic DNA ligase as contemplated by the disclosure can be isolated from a recombinant source and are thermostable and can withstand PCR conditions. In a preferred embodiment, the 9° N DNA Ligase from New England BioLabs is used which is active at elevated temperatures.

In one embodiment, the ligation product comprises the sequence of interest. In another embodiment, the sequencing adapters are added to 5′ and 3′ ends of the ligation product by ligation. In one embodiment, the sequence of interest contains an SNP. In another embodiment, the sequence of interest contains a mutation, a deletion or an insertion. In one embodiment, the adapter-ligated library DNA is PCR amplified prior to sequencing. In another embodiment, the steps are performed directly in a cell culture or tissue sample and the resulting sequencing libraries are amplified by in situ PCR. In yet another embodiment, the cell and tissue samples are fixed.

The present disclosure provides a composition for preparing a sequencing library from a target DNA comprising a first enzyme comprising an endonuclease, a first nucleotide sequence comprising a first guide RNA, a second nucleotide sequence comprising a second guide RNA, a second enzyme comprising a ligase, a third nucleotide sequence comprising a first sequencing adapter, a fourth nucleotide sequence comprising a second sequencing adapter, and a buffer comprising a solution in which both the endonuclease and ligase are active. In one embodiment, the first and second RNAs guide the endonuclease to specific sites flanking regions of interest in the DNA wherein the endonuclease cleaves the DNA in a site specific manner. In one embodiment, composition further comprises a buffer for stabilizing the nucleotide sequences and the enzymes.

The present disclosure further provides a kit for preparing a sequencing library from a target DNA comprising the composition of a first enzyme comprising an endonuclease, a first nucleotide sequence comprising a first guide RNA, a second nucleotide sequence comprising a second guide RNA, a second enzyme comprising a ligase, a third nucleotide sequence comprising a first sequencing adapter, a fourth nucleotide sequence comprising a second sequencing adapter, and a buffer comprising a solution in which both the endonuclease and ligase are active and and a reagent for reconstitution and/or dilution. In one embodiment, the kit further comprises a control reagent.

### Cas9 Description

RNA guided DNA binding proteins are readily known to those of skill in the art to bind to DNA for various purposes. Such DNA binding proteins may be naturally occurring. DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February, 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June, 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February, 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June, 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January, 2011) each of which are hereby incorporated by reference in their entireties.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAil; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is provided in Deltcheva et al., Nature 471, 602-607 (2011) hereby incorporated by reference in its entirety.

According to certain aspects of the disclosure, any nucleic-acid guided or programmable endonuclease that can be heat inactivated at 98° C. can be used. Modification to the Cas9 protein is also contemplated by the present disclosure. Cas9 orthologs (e.g., NM-Cas9, ST1-Cas9), engineered Cas9 variants (e.g., eCas9, Cas9-HF1), and other cas family RNA-guided endonucleases (e.g., Cpf1) are contemplated which provide means of addressing a larger target site space.

According to certain aspects, the DNA binding protein is altered or otherwise modified to inactivate the nuclease activity. Such alteration or modification includes altering one or more amino acids to inactivate the nuclease activity or the nuclease domain Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase. An exemplary DNA binding protein is an RNA guided DNA binding protein nuclease of a Type II CRISPR System, such as a Cas9 protein or modified Cas9 or homolog of Cas9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null or nuclease deficient Cas9 protein.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinek et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. thermophiles or S. pyogenes and protein sequences having at least 30%, 40%, 50%, 60%, 70%, 80%, 90%, 95%, 98% or 99% homology thereto and being a DNA binding protein, such as an RNA guided DNA binding protein.

CRISPR systems useful in the present disclosure are described in R. Banangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

An exemplary CRISPR system includes the S. thermophiles Cas9 nuclease (ST1 Cas9) (see Esvelt K M, et al., Orthogonal Cas9 proteins for RNA-guided gene regulation and editing, Nature Methods., (2013) hereby incorporated by reference in its entirety). An exemplary CRISPR system includes the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). According to certain aspects, a nuclease null or nuclease deficient Cas 9 can be used in the methods described herein. Such nuclease null or nuclease deficient Cas9 proteins are described in Gilbert, L. A. et al. CRISPR-mediated modular RNA-guided regulation of transcription in eukaryotes. Cell 154, 442-451 (2013); Mali, P. et al. CAS9 transcriptional activators for target specificity screening and paired nickases for cooperative genome engineering. Nature biotechnology 31, 833-838 (2013); Maeder, M. L. et al. CRISPR RNA-guided activation of endogenous human genes. Nature methods 10, 977-979 (2013); and Perez-Pinera, P. et al. RNA-guided gene activation by CRISPR-Cas9-based transcription factors. Nature methods 10, 973-976 (2013) each of which are hereby incorporated by reference in its entirety. The DNA locus targeted by Cas9 (and by its nuclease-deficient mutant, “dCas9” precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 or a nuclease deficient Cas9 to a target nucleic acid. In a multitude of CRISPR-based biotechnology applications (see Mali, P., Esvelt, K. M. & Church, G. M. Cas9 as a versatile tool for engineering biology. Nature methods 10, 957-963 (2013); Hsu, P. D., Lander, E. S. & Zhang, F. Development and Applications of CRISPR-Cas9 for Genome Engineering. Cell 157, 1262-1278 (2014); Chen, B. et al. Dynamic imaging of genomic loci in living human cells by an optimized CRISPR/Cas system. Cell 155, 1479-1491 (2013); Shalem, O. et al. Genome-scale CRISPR-Cas9 knockout screening in human cells. Science 343, 84-87 (2014); Wang, T., Wei, J. J., Sabatini, D. M. & Lander, E. S. Genetic screens in human cells using the CRISPR-Cas9 system. Science 343, 80-84 (2014); Nissim, L., Perli, S. D., Fridkin, A., Perez-Pinera, P. & Lu, T. K. Multiplexed and Programmable Regulation of Gene Networks with an Integrated RNA and CRISPR/Cas Toolkit in Human Cells. Molecular cell 54, 698-710 (2014); Ryan, O. W. et al. Selection of chromosomal DNA libraries using a multiplex CRISPR system. eLife 3 (2014); Gilbert, L. A. et al. Genome-Scale CRISPR-Mediated Control of Gene Repression and Activation. Cell (2014); and Citorik, R. J., Mimee, M. & Lu, T. K. Sequence-specific antimicrobials using efficiently delivered RNA-guided nucleases. Nature biotechnology (2014) each of which are hereby incorporated by reference in its entirety), the guide is often presented in a so-called sgRNA (single guide RNA), wherein the two natural Cas9 RNA cofactors (gRNA and tracrRNA) are fused via an engineered loop or linker.

The disclosure provides that the endonucleases and ligases may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

The disclosure provides that the Cas9 protein is exogenous to the cells or tissues. The disclosure provides that the Cas9 protein is foreign to the cells or tissues. The disclosure provides that the Cas9 protein is non-naturally occurring within the cell.

### Guide RNA Description

Embodiments of the present disclosure are directed to the use of a CRISPR/Cas system and, in particular, a guide RNA which may include one or more of a spacer sequence, a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

According to certain aspects, the guide RNA is between about 10 to about 500 nucleotides. According to one aspect, the guide RNA is between about 20 to about 100 nucleotides. According to certain aspects, the spacer sequence is between about 10 and about 500 nucleotides in length. According to certain aspects, the tracr mate sequence is between about 10 and about 500 nucleotides in length. According to certain aspects, the tracr sequence is between about 10 and about 100 nucleotides in length. According to certain aspects, the linker nucleic acid sequence is between about 10 and about 100 nucleotides in length.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence (if present). According to this aspect, all of the ranges for the spacer sequence, tracr mate sequence, tracr sequence, and linker sequence (if present) are incorporated herein by reference and need not be repeated. A guide RNA as described herein may have a total length based on summing values provided by the ranges described herein. Aspects of the present disclosure are directed to methods of making such guide RNAs as described herein by expressing constructs encoding such guide RNA using promoters and terminators and optionally other genetic elements as described herein.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

### Target Nucleic Acid Sequence

A target nucleic acid sequence includes any nucleic acid sequence, such as a genomic nucleic acid sequence or a gene to which a Cas9 pre-complexed with one or more pairs of fragmentation gRNAs as described herein can be useful to either cut, nick or regulate. Target nucleic acids include nucleic acid sequences capable of being expressed into proteins. The disclosure provides that the target nucleic acid is mammalian genomic DNA, human genomic DNA, mitochondrial DNA, plasmid DNA, bacterial and viral DNA, exogenous DNA or cellular RNA.

### Cells and Tissues

Cells and tissues according to the present disclosure include any cell or tissue into which foreign nucleic acids can be introduced and expressed as described herein. It is to be understood that the basic concepts of the present disclosure described herein are not limited by cell or tissue type. Cells according to the present disclosure include eukaryotic cells, prokaryotic cells, animal cells, plant cells, fungal cells, archael cells, eubacterial cells and the like. Cells include eukaryotic cells such as yeast cells, plant cells, and animal cells. Particular cells include mammalian cells. Further, cells include any in which it would be beneficial or desirable to cut, nick or regulate a target nucleic acid. Tissues according to the present disclosure include nervous, connective, epithelial, and muscular tissues. Such cells and tissues may include those which are deficient in expression of a particular protein leading to a disease or detrimental condition. Such diseases or detrimental conditions are readily known to those of skill in the art. According to the present disclosure, the nucleic acid responsible for expressing the particular protein may be targeted by the methods described herein and a transcriptional activator resulting in upregulation of the target nucleic acid and corresponding expression of the particular protein. In this manner, the methods described herein provide therapeutic treatment. Such cells may include those which over express a particular protein leading to a disease or detrimental condition. Such diseases or detrimental conditions are readily known to those of skill in the art. According to the present disclosure, the nucleic acid responsible for expressing the particular protein may be targeted by the methods described herein and a transcriptional repressor resulting in downregulation of the target nucleic acid and corresponding expression of the particular protein. In this manner, the methods described herein provide therapeutic treatment.

In one embodiment, the cells and tissues of the present disclosure are human cells and tissues. In another embodiment, the cell is a stem cell whether adult or embryonic. In one embodiment, the cell is a pluripotent stem cell. In one embodiment, the cell is an induced pluripotent stem cell. In one embodiment, the cell is a human induced pluripotent stem cell. In one embodiment, the cell is in vitro, in vivo or ex vivo.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Application of Single Tube Cas9 Library Preparation to SNP Detection in Bacterial DNA and Comparison to Traditional Targeted PCR Library Preparation

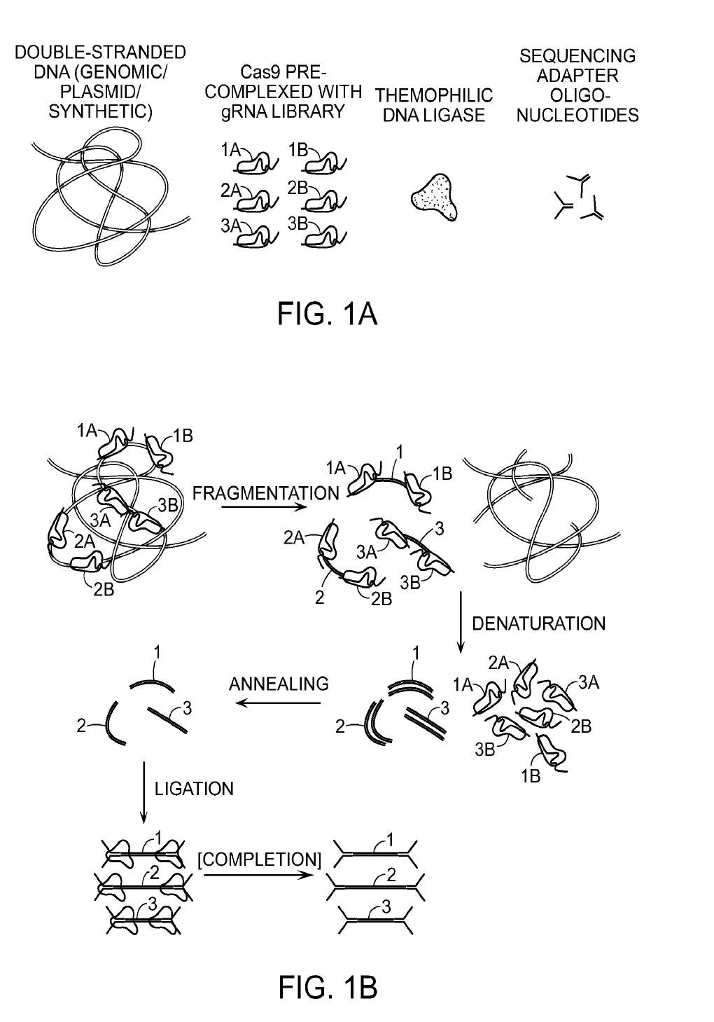

Preparing a sequencing library from a target DNA includes the following minimum compositions: double stranded target DNA (genomic/plasmid/synthetic), Cas9 pre-complexed with one or more pairs of fragmentation gRNAs, a thermophilic DNA ligase, and application-specific adapter oligonucleotides (FIG. 1A). The gRNAs guide the Cas9 endonuclease to specific sites flanking regions of interest in the target DNA (FIG. 1B). The mixture is subjected to the following sequential steps of thermal cycling delineated by temperature. At 37° C., the pre-complexed Cas9-gRNA holoenzymes catalyze the selective fragmentation of the target DNA. Denaturation at 95° C. removes Cas9 from the fragmented DNA and subsequent cooling allows for the nucleic acids to properly anneal. Continuation of the reaction at 45° C. allows the thermophilic ligase to catalyze the ligation of adapter oligonucleotides onto the DNA fragments (FIG. 1C).

As a proof of concept, single tube Cas9 library preparation was used to determine the frequency of a single nucleotide polymorphism (SNP) known to confer resistance to the common antibiotic rifampicin within a population of resistance E. coli cells. Rifampicin is a widely-used antibiotic that inhibits RNA polymerase function, and there are a number of well-characterized mutations within the E. coli rpoB gene that perturb its mechanism of action, conferring resistance to the cell. In both clinical and academic settings, it is desirable to rapidly, sensitively, and inexpensively characterize the identities and frequencies of such mutations known to confer resistance to antibiotics (to inform drug development, treatment decisions, or research hypotheses), and next-generation sequencing is a common means of doing so.

In this experiment, cells from a population known to harbor resistance to rifampicin were subjected to lysis by lithium acetate (LiOAc) and subsequent DNA extraction. Briefly, cells were scraped from a 100 mm LB agar plate and added to tube containing 300 μl of 200 mM LiOAc+1% SDS, vortexed briefly, and incubated at 70° C. for 10 minutes. After incubation, 900 μl of 95% ethanol was added to precipitate DNA, samples were vortexed briefly, and then centrifuged at 13,000 RCF for 3 minutes to pellet DNA and cellular debris. The resulting supernatant was discarded and pellets were washed once by addition of 500 μl of 70% ethanol followed by a 5 minute spin at 13,000 RCF. The supernatant was again discarded and residual ethanol was removed with a pipet. Tubes were allowed to sit at room temperature with their caps open for 5 minutes to remove any remaining ethanol. Genomic DNA was resuspended in 100 μl of TE and then quantified on a Nanodrop 2000 spectrophotometer.

The quantified genomic DNA was then used as an input for both single tube Cas9 library preparation and for traditional targeted PCR library preparation. In both cases, five separate technical replicates were provided at the point of initial mixture composition, as described below.

In the case of the single tube Cas9 library preparation, 50 ng of the purified genomic DNA was added to a tube containing the following reagents: 2 ul of 10×C9L buffer, 2 ul of 9° N ligase (NEB #M0238), 1 ul of Cas9 nuclease (NEB #M0386S), 3 ul of 300 nM sgRNA L (TCTGGATACCCTGATGCCACGTTTTAGAGCTAGAAATAGCAAGTTAAAATAAGG CTAGTCCGTTATCAACTTGAAAAAGTGGCACCGAGTCGGTGCTTTTT), 3 ul of 300 nM sgRNA R (TTCGTTAGTCTGTGCGTACAGTTTTAGAGCTAGAAATAGCAAGTTAAAATAAGG CTAGTCCGTTATCAACTTGAAAAAGTGGCACCGAGTCGGTGCTTTTT), 4 ul of adapter oligonucleotide mix, and nuclease free water to 20 ul. The mixture was placed in a thermocycler and heated to 37° C. for 45 minutes to allow for Cas9 digestion at the target sites. The mixture was then heated to 98° C. for 10 minutes to denature the Cas9 protein, and then cooled to 45° C. for 45 minutes to allow for renaturation of the target DNA fragments and adapter oligonucleotides, and subsequent ligation of the adapter oligonucleotides onto the target DNA fragments by the thermophilic ligase 9° N. The resulting solution was used as the direct input for indexing PCR as described below.

In the case of the targeted PCR library preparation, 50 ng of the purified genomic DNA was added to a tube containing the following reagents: 4 ul of 5× Phusion HF buffer (NEB #M0530L), 0.4 ul 10 mM dNTPs (NEB # N0447L), 0.1 ul 10 uM forward primer (CTTTCCCTACACGACGCTCTTCCGATCTGATCTGGATACCCTGATGCCACAG), 0.1 ul 10 uM reverse primer (GGAGTTCAGACGTGTGCTCTTCCGATCTTTAGTCTGTGCGTACACGGACAGAGA G), 0.2 ul Phusion DNA polymerase (NEB #M0530L) and nuclease water to a final volume of 20 ul. The mixture was then placed in a thermocycler and subjected to denaturation at 98° C. for 30 seconds, followed by 30 cycles of 98° C. denaturation for 5 seconds, 60° C. annealing for 15 seconds, and 72° C. extension for 15 seconds. The mixture was then subjected to a final extension at 72° C. for 5 minutes. Finally, the mixture was purified using the Qiagen QIAquick PCR Purification kit (Qiagen #28104).

The outputs of the two respective preparation pipelines were used as the input for indexing PCR using the NEBNext Multiplex Oligos, according to the manufacturer's instructions (NEB #E7335S). This adds the remaining adapter sequence and barcodes necessary for sequencing and demultiplexing on the Illumina line of sequencing devices. The resulting pool of indexing libraries was subjected to 300 rounds of sequencing on the Illumina MiSeq, using the 300 cycle v2 reagent kit (Illumina #MS-102-2002). The demultiplexed FASTQ files resulting from the sequencing run were then aligned to the E. coli rpoB gene reference sequence using the Bowtie2 2.2.6 aligner (Langmead B, Salzberg S. Fast gapped-read alignment with Bowtie 2. Nature Methods. 2012, 9:357-359). The frequency of the 1534T>C mutation was then determined using a custom Python script.

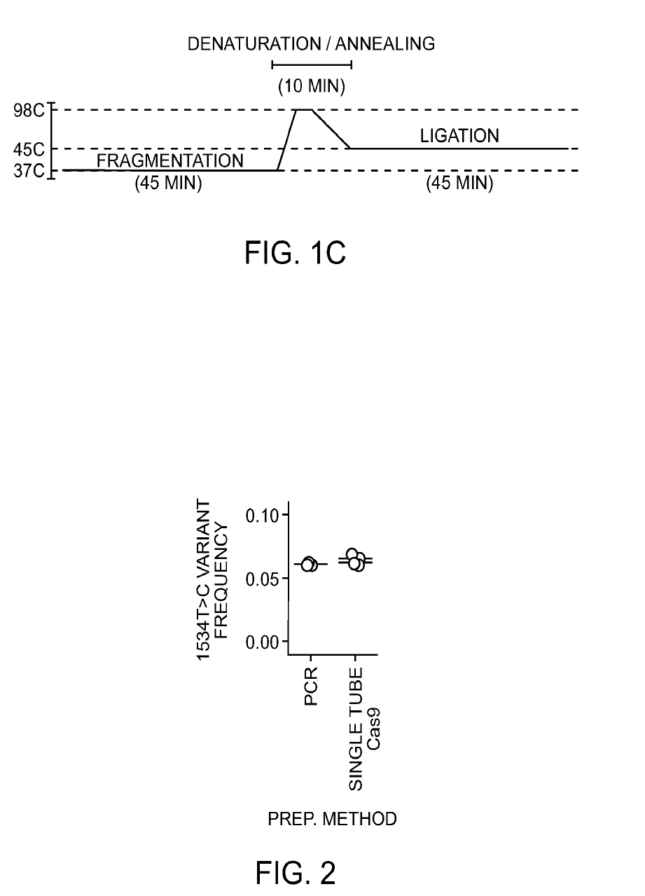

Raw data of the 5 independent technical replicates for each preparation method are summarized in Table 1 and 1534T>C variant frequency detected from direct PCR based library preparation and single tube Cas9 library preparation are shown in (FIG. 2). (n=5 independent technical replicates, error bars are S.E.M.).

TABLE 1

| Prep method | Rep. 1 | Rep. 2 | Rep. 3 | Rep. 4 | Rep. 5 | Mean | S.E.M. |
|---|---|---|---|---|---|---|---|
| PCR | 0.0615 | 0.0601 | 0.0613 | 0.0619 | 0.0607 | 0.0611 | 0.000323 |
| Single tube Cas9 | 0.0631 | 0.0656 | 0.0605 | 0.0582 | 0.0614 | 0.0618 | 0.00123 |

### PCR Scheme:

1. PCR primers were designed to flank the primary mutational hotspot within rpoB. These primers additionally contain 5′ adapter sequence amenable to further indexing and sequencing on the Illumina sequencing platform.

| Illumina adapter sequence |
F Illumina adapter sequence:
CTTTCCCTACACGACGCTCTTCCGATCT
R Illumina adapter sequence:
GGAGTTCAGACGTGTGCTCTTCCGATCT
F primer: [F Illumina adapter sequence]
GATCTGGATACCCTGATGCCACAG
R primer: [R Illumina adapter sequence]
TTAGTCTGTGCGTACACGGACAGAGAG

2. PCR reactions were prepared as follows:

a. 50 ng of genomic DNA

b. 4 ul 5× Phusion HF buffer (NEB #M0530L)

c. 0.4 ul 10 mM dNTPs (NEB # N0447L)

d. 0.1 ul 10 uM forward primer

e. 0.1 ul 10 uM reverse primer

f. 0.2 ul Phusion DNA polymerase (NEB #M0530L)

g. Nuclease-free water to 20 ul

3. PCR cycling was performed as follows:

98° C. for 30 seconds

30 cycles of:

98° C. for 5 seconds

60° C. for 15 seconds

72° C. for 15 seconds

72° C. for 5 minutes

4° C. hold

4. PCR reactions were purified by Qiagen QIAquick PCR Purification (Qiagen #28104) columns in accordance with the manufacturer's instructions.
5. 1 ul of each reaction was used directly as input for indexing and sequencing on an Illumina Miseq.

### Single Tube Cas9 Scheme:

1. The following sgRNAs were produced by in vitro transcription:

L: TCTGGATACCCTGATGCCAC [sgRNA tail]
R: TTCGTTAGTCTGTGCGTACA [sgRNA tail]
sgRNA tail:
GTTTTAGAGCTAGAAATAGCAAGTTAAAATAAGGCTAGTCCGTTATCAAC
TTGAAAAAGTGGCACCGAGTCGGTGCTTTTT

2. Reactions were prepared as follows:
a. 50 ng of genomic DNA
b. 2 ul of 10×C9L buffer
c. 2 ul of 9° N ligase (NEB #M0238)
d. 1 ul of Cas9 nuclease (NEB #M0386S)
e. 3 ul of 300 nM sgRNA L (see above)
f. 3 ul of 300 nM sgRNA R (see above)
g. 4 ul of adapter oligonucleotide mix
h. Nuclease-free water to 20 ul

3. Reaction cycling was performed as follows:
37° C. for 45 minutes
98° C. for 10 minutes
45° C. for 45 minutes

4. 1 ul of each reaction was used directly as input for indexing and sequencing on an Illumina Miseq.

### Example II

### Application of Single Tube Cas9 Library Preparation to SNP Detection in Human Genomic DNA

Human genomic DNA is extracted from a tumor biopsy or other clinical tissue isolate using well known methods, such as a silica-membrane based nucleic acid purification kit (e.g., the QIAamp DNA mini kit, #51304). The genomic DNA is then quantified using spectrophotometric or fluorescent assay, as is well known to those skilled in the art. The genomic DNA is then added to a single tube Cas9 library preparation solution containing a plurality of single guide RNAs (sgRNAs) suitable for targeting SNPs of diagnostic interest. For example, a panel of sgRNAs designed to target SNPs within the BRCA1 gene that confer prognostic power with regard to breast cancer diagnosis may be employed:

| refSNP | BRCA1 | | |
|---|---|---|---|
| ID | substitution | L spacer sequence | R spacer sequence |
| rs1799950 | Q356R | GACTCCCAGCACAGAAAAAA | ACCTAACAGTTCATCACTTC |
| rs4986850 | D693N | GAAGGTAAAGAACCTGCAAC | TTTTCTTCTCTTGGAAGGCT |
| rs2227945 | S1140G | AAGTTATCTGAAATCAGATA | TTGGCTCAGGGTTACCGAAG |
| rs16942 | K1183R | (Same as rs2227945 L) | (Same as rs2227945 R) |
| rs1799966 | S1613G | TTCAGAGGGAACCCCTTACC | TATGAGCAGCAGCTGGACTC |

In the above table, the spacer region of each guide pair for a given target SNP is provided. All spacers are part of sgRNAs with the tail sequence provided in EXAMPLE 1. Note that in some cases two or more SNPs may be targeted by the same sgRNA pair (see r222745 and r16942, above). A 300 nM solution containing all of the described sgRNAs may be prepared and compose the single tube Cas9 library preparation solution as follows:
a. 50 ng of human genomic DNA
b. 2 ul of 10×C9L buffer
c. 2 ul of 9° N ligase (NEB #M0238)
d. 1 ul of Cas9 nuclease (NEB #M0386S)
f. 6 ul of 300 nM sgRNA mixture (see above)
g. 4 ul of adapter oligonucleotide mix
h. Nuclease-free water to 20 ul

Components b-g may be prepared as a 2× solution (using components f+g at higher concentration) to be used to process many input samples, and such a solution would be diluted to a 1× working concentration at the time at which the genomic DNA, component a, is added (with component h, nuclease free water, being the diluent).

The libraries prepared using the aforementioned sgRNAs in a single tube Cas9 library preparation reaction may then be interrogated by common sequencing or hybridization reactions known to those skilled in the art, such as next-generation sequencing. A bioinformatics pipeline may then be utilized to determine the prevalence and frequency of any targeted SNPs, in such a manner that heterozygosity may be resolved.

### Example III

### Application of Single Tube Cas9 Library Preparation to an In Situ Sample

A biological specimen is fixed and permeabilized using well known methods, such by treatment with formaldehyde followed by detergent to remove the lipid membranes. The sample may be subjected to additional treatments, known to those familiar with the art, for the purpose of rendering the nucleic acids, such as genomic DNA, both stabilized in space and accessible to biochemical reactions. For example, the DNA may be modified with linkers for covalent attachment into a hydrogel matrix, and such a hydrogel matrix synthesized in situ. The sample may then be further permeabilized and nucleic acids de-protected from bound proteins by means of treatment which disrupts protein structure, such as digestion with proteinases and denaturation with SDS, urea, and/or guanidine salt. A reaction mixture containing Cas9 (pre-complexed with a plurality of sgRNAs), a thermophilic DNA ligase (e.g., 9° N), and adapter oligos, (as described in Examples 1+2, above) is added to the sample such that the genomic DNA is cleaved by the targeted endonucleases at specific sites and ligated to the adapter oligos in situ. The adapter-modified fragments, which contain genomic sequences of interest, are then amplified using methods well known to those familiar with the field, such as in situ polony PCR (Shendure Science 2005) or isothermal amplification (Ma PNAS 2013). The in situ clonally amplified sequencing templates are then sequenced in situ using sequencing by hybridization, sequencing by synthesis by polymerase, or sequencing by ligation, to detect the genomic sequence.

What is claimed is:

1. A method of preparing a sequencing library from a target DNA comprising the steps of:
contacting the DNA with a composition comprising an endonuclease, a first guide RNA, a second guide RNA, a ligase, and sequencing adapters, wherein the first and second RNAs guide the endonuclease to specific sites flanking regions of interest in the DNA,
subjecting the DNA and the composition to thermal cycling to allow cleavage of the DNA at the sites flanking the regions of interest by the endonuclease, and
subjecting the DNA and the composition to a temperature to allow ligation of the cleaved DNA fragments including the regions of interest with the sequencing adapters to generate a sequencing library.

2. The method of claim 1 wherein the target DNA is mammalian genomic DNA.

3. The method of claim 1 wherein the target DNA is human genomic DNA.

4. The method of claim 1 wherein the target DNA is bacterial genomic DNA.

5. The method of claim 1 wherein the target DNA is synthetic DNA.

6. The method of claim 5 wherein the synthetic DNA is in the form of transfected or integrated library.

7. The method of claim 1 wherein the first and second guide RNAs are complementary to sequences flanking the regions of interest in the DNA.

8. The method of claim 1 wherein the endonuclease comprises Cas9, Cas9 orthologs or engineered Cas9 variants.

9. The method of claim 8 wherein the Cas9 orthologs comprise NM-/ST1-Cas9 and Cpf1.

10. The method of claim 8 wherein the engineered Cas9 variants comprise eCas9 and Cas9-HF1.

11. The method of claim 1 wherein the sequencing adapters are added to 5′ and 3′ ends of the cleaved DNA fragments by ligation.

12. The method of claim 1 wherein the ligase is a thermophilic DNA ligase.

13. The method of claim 1 wherein a plurality of sequencing libraries are prepared from a plurality of target DNAs.

14. The method of claim 1 wherein the steps are performed directly in a cell culture or tissue sample and the resulting sequencing libraries are amplified by in situ PCR.

15. The method of claim 14 wherein the cell and tissue samples are fixed.

16. A method of determining a sequence of interest in a target DNA comprising the steps of:
contacting the DNA with a composition comprising an endonuclease, a first guide RNA, a second guide RNA, a ligase, and sequencing adapters, wherein the first and second RNAs guide the endonuclease to sites flanking the sequence of interest in the DNA,
subjecting the DNA and the composition to thermal cycling to allow cleavage of the DNA at sites flanking the sequence of interest by the endonuclease,
subjecting the DNA and the composition to a temperature to allow ligation of the cleaved DNA fragment including the sequence of interest with the sequencing adapters to generate a ligation product, and
sequencing the ligation product to determine the sequence of interest.

17. The method of claim 16 wherein the target DNA is mammalian genomic DNA.

18. The method of claim 16 wherein the target DNA is human genomic DNA.

19. The method of claim 16 wherein the target DNA is bacterial genomic DNA.

20. The method of claim 16 wherein the target DNA is synthetic DNA.

21. The method of claim 20 wherein the synthetic DNA is in the form of transfected or integrated library.

22. The method of claim 16 wherein the first and second guide RNAs comprising complementary sequences to the sequences flanking the sequence of interest in the DNA.

23. The method of claim 16 wherein the endonuclease comprises Cas9, Cas9 orthologs or engineered Cas9 variants.

24. The method of claim 23 wherein the Cas9 orthologs comprise NM-/ST1-Cas9 and Cpf1.

25. The method of claim 23 wherein the engineered Cas9 variants comprise eCas9 and Cas9-HF1.

26. The method of claim 16 wherein the ligation product comprises the sequence of interest.

27. The method of claim 16 wherein the sequencing adapters are added to 5′ and 3′ ends of the ligation product by ligation.

28. The method of claim 16 wherein the ligase is a thermophilic DNA ligase.

29. The method of claim 16 wherein a plurality of sequence of interest in the DNA are detected.

30. The method of claim 16 wherein the sequence of interest contains an SNP.

31. The method of claim 16 wherein the sequence of interest contains a mutation, a deletion or an insertion.

32. The method of claim 16 wherein the adapter-ligated library DNA is PCR amplified prior to sequencing.

33. The method of claim 16 wherein the steps are performed directly in a cell culture or tissue sample and the resulting sequencing libraries are amplified by in situ PCR.

34. The method of claim 33 wherein the cell and tissue samples are fixed.

35. A composition for preparing a sequencing library from a target DNA comprising
a first enzyme comprising an endonuclease,
a first nucleotide sequence comprising a first guide RNA,
a second nucleotide sequence comprising a second guide RNA,
a second enzyme comprising a ligase,
a third nucleotide sequence comprising a first sequencing adapter,
a fourth nucleotide sequence comprising a second sequencing adapter, and
a buffer comprising a solution in which both the endonuclease and ligase are active.

36. The composition of claim 35 wherein the target DNA is mammalian genomic DNA.

37. The composition of claim 35 wherein the target DNA is human genomic DNA.

38. The composition of claim 35 wherein the target DNA is bacterial genomic DNA.

39. The composition of claim 35 wherein the target DNA is synthetic DNA.

40. The composition of claim 39 wherein the synthetic DNA is in the form of transfected or integrated library.

41. The composition of claim 35 wherein the first and second RNAs guide the endonuclease to specific sites flanking regions of interest in the DNA wherein the endonuclease cleaves the DNA in a site specific manner.

42. The composition of claim 35 wherein the first and second guide RNAs are complementary to sequences flanking the regions of interest in the DNA.

43. The composition of claim 35 wherein the endonuclease comprises Cas9, Cas9 orthologs or engineered Cas9 variants.

44. The composition of claim 43 wherein the Cas9 orthologs comprise NM-/ST1-Cas9 and Cpf1.

45. The composition of claim 43 wherein the engineered Cas9 variants comprise eCas9 and Cas9-HF1.

46. The composition of claim 35 wherein the first and second sequencing adapters are added to 5′ and 3′ ends of the cleaved DNA fragments by ligation.

47. The composition of claim 35 wherein the ligase is a thermophilic DNA ligase.

48. The composition of claim 35 further comprising a buffer for stabilizing the nucleotide sequences and the enzymes.

49. A kit for preparing a sequencing library from a target DNA comprising
the composition of claim 35, and
a reagent for reconstitution and/or dilution.

50. The kit of claim 49 further comprising a control reagent.


SEQUENCE LISTING (19 sequences)

SEQ ID NO: 1 - DNA, 101 bp, Artificial Sequence
  Description of Artificial Sequence Synthetic polynucleotide
tctggataccctgatgccacgttttagagctagaaatagcaagttaaaataaggctagtccgttatcaacttgaaaaagtggcaccgagtcggtgcttttt

SEQ ID NO: 2 - DNA, 101 bp, Artificial Sequence
  Description of Artificial Sequence Synthetic polynucleotide
ttcgttagtctgtgcgtacagttttagagctagaaatagcaagttaaaataaggctagtccgttatcaacttgaaaaagtggcaccgagtcggtgcttttt

SEQ ID NO: 3 - DNA, 52 bp, Artificial Sequence
  Description of Artificial Sequence Synthetic primer
ctttccctacacgacgctcttccgatctgatctggataccctgatgccacag

SEQ ID NO: 4 - DNA, 55 bp, Artificial Sequence
  Description of Artificial Sequence Synthetic primer
ggagttcagacgtgtgctcttccgatctttagtctgtgcgtacacggacagagag

SEQ ID NO: 5 - DNA, 28 bp, Artificial Sequence
  Description of Artificial Sequence Synthetic primer
ctttccctacacgacgctcttccgatct

SEQ ID NO: 6 - DNA, 28 bp, Artificial Sequence
  Description of Artificial Sequence Synthetic primer
ggagttcagacgtgtgctcttccgatct

SEQ ID NO: 7 - DNA, 24 bp, Artificial Sequence
  Description of Artificial Sequence Synthetic primer
gatctggataccctgatgccacag

SEQ ID NO: 8 - DNA, 27 bp, Artificial Sequence
  Description of Artificial Sequence Synthetic primer
ttagtctgtgcgtacacggacagagag

SEQ ID NO: 9 - DNA, 20 bp, Artificial Sequence
  Description of Artificial Sequence Synthetic oligonucleotide
tctggataccctgatgccac

SEQ ID NO: 10 - DNA, 20 bp, Artificial Sequence
  Description of Artificial Sequence Synthetic oligonucleotide
ttcgttagtctgtgcgtaca

SEQ ID NO: 11 - DNA, 81 bp, Artificial Sequence
  Description of Artificial Sequence Synthetic oligonucleotide
gttttagagctagaaatagcaagttaaaataaggctagtccgttatcaacttgaaaaagtggcaccgagtcggtgcttttt

SEQ ID NO: 12 - DNA, 20 bp, Artificial Sequence
  Description of Artificial Sequence Synthetic oligonucleotide
gactcccagcacagaaaaaa

SEQ ID NO: 13 - DNA, 20 bp, Artificial Sequence
  Description of Artificial Sequence Synthetic oligonucleotide
gaaggtaaagaacctgcaac

SEQ ID NO: 14 - DNA, 20 bp, Artificial Sequence
  Description of Artificial Sequence Synthetic oligonucleotide
aagttatctgaaatcagata

SEQ ID NO: 15 - DNA, 20 bp, Artificial Sequence
  Description of Artificial Sequence Synthetic oligonucleotide
ttcagagggaaccccttacc

SEQ ID NO: 16 - DNA, 20 bp, Artificial Sequence
  Description of Artificial Sequence Synthetic oligonucleotide
acctaacagttcatcacttc

SEQ ID NO: 17 - DNA, 20 bp, Artificial Sequence
  Description of Artificial Sequence Synthetic oligonucleotide
ttttcttctcttggaaggct

SEQ ID NO: 18 - DNA, 20 bp, Artificial Sequence
  Description of Artificial Sequence Synthetic oligonucleotide
ttggctcagggttaccgaag

SEQ ID NO: 19 - DNA, 20 bp, Artificial Sequence
  Description of Artificial Sequence Synthetic oligonucleotide
tatgagcagcagctggactc